(19) United States
(12) Patent Application Publication
(10) Pub. No.: US 2006/0008858 A1
(43) Pub. Date: Jan. 12, 2006

(54) Osteoblast factor(s) that regulates human prostate cancer migration to and invasion of bone

(75) Inventors: Christopher L. Hall, Ypsilanti, MI (US); Michael W. Long, Northville, MI (US)

Correspondence Address: FULBRIGHT & JAWORSKI L.L.P., 600 CONGRESS AVE., SUITE 2400, AUSTIN, TX, 78701, US

(73) Assignee: The Regents of the University of Michigan

(21) Appl. No.: 11/046,666

(22) Filed: Jan. 28, 2005

Related U.S. Application Data
(60) Provisional application No. 60/539,887, filed on Jan. 28, 2004.

(51) Int. Cl. G01N 33/574; C07H 21/04; C07K 14/82; C07K 16/30

(52) U.S. Cl. 435/7.23; 435/69.3; 435/320.1; 435/325; 530/350; 530/388.8; 536/23.5

(57) ABSTRACT

Human osteogenic cells secrete biological activities that stimulate cancer cells to migrate to and/or invade tissues. Conditioned medium (CM) produced by human pre-osteoblasts and osteoblasts induced migration and tissue invasion of human prostate cancer cells. Thus, conditioned media and/or proteins isolated therefrom may be used to identify metastasis-inducing factors. Also described are methods of identifying an inhibitor of such factors.

This application claims benefit of priority to U.S. Provisional Application Ser. No. 60/539,887, filed Jan. 28, 2004, the entire contents of which are hereby incorporated by reference. The government owns rights in the present invention pursuant to grant number R01 DK061456.

### BACKGROUND OF THE INVENTION

1. Field of the Invention

The present invention relates generally to the fields of biology and medicine. More particularly, it concerns a process for the identification, isolation and use of factors from preosteoblasts and osteoblasts that stimulate metastasis of prostate cancer cell to bone.

2. Description of Related Art

Prostate cancer (CaP) is the second leading cause of cancer death in men with an estimated 189,000 men/year diagnosed as having prostate cancer. It is notable that 83% of all prostate cancers are discovered in local and regional stages. In 2002, an estimated 30,200 men are expected to die of prostate cancer.

A unique clinical feature of certain cancers is the formation of osteoblastic or bone-producing lesions in the pelvis and vertebral column, in which large amounts of bone are generated at the site of CaP metastasis. In fact, spine metastasis represents 90% of prostate cancer metastasis, and recurrence is common (45% risk within 2 years). However, despite significant research efforts, the molecular mechanism mediating this osteoblastic response as yet is unclear.

Current therapies consist of surgical intervention, radio-therapy, hormone therapy, and chemotherapy. All of these have extensive side effects, and are directed at eradication of the primary tumor, often missing metastatic lesions, or not preventing this process. Thus, there remains a need to identify and utilize factors that are responsible for bone metastasis in the identification of new and improved methods of therapy for this disease.

### SUMMARY OF THE INVENTION

Thus, in accordance with the present invention, there are provided methods of identifying an agent useful in preventing cancer cell metastasis to bone comprising (a) providing osteogenic precursor cell-conditioned medium (OCM); (b) providing a bone-metastatic cancer cell; (c) contacting the OCM with the bone-metastatic cancer cell in the presence of a candidate substance; and (d) assessing the migration and/or invasion of the bone-metastatic cancer cell, wherein a difference in the migration and/or invasion seen in step (d), as compared to the migration and/or invasion seen in the absence of the candidate substance, identifies the candidate substance as a bone metastasis inhibitor. The candidate substance may be an organopharmaceutical small molecule, an antibody or fragment thereof, or a peptide, polypeptide, or peptidomimetic. The bone-metastatic cancer cell may be a prostate cancer cell, such as a LNCaP-C4-2B cell, or a breast cancer cell. The cells may be primary tumor isolates or tumor cell lines. The OCM may be conditioned with a preosteoblast cell, an osteoblast cell, an osteoblast precursor cell, and/or an osteoblast accessory cell. The method may further comprise performing step (d) in the absence of the candidate substance.

In another embodiment, there is provided a composition comprising medium conditioned by growth of an osteogenic cell therein. Also provided is a protein factor obtained from osteogenic cell-conditioned medium that promotes migration to and/or invasion of bone tissue by metastatic prostate cancer cells. In yet another embodiment, there is provided a heat-labile protein factor obtained from osteogenic cell-conditioned medium that promotes migration to and/or invasion of bone tissue by prostate cancer cells. In still yet another embodiment, there are provided methods of obtaining a protein factor produced by an osteogenic cell, wherein the factor promotes migration to and/or invasion of bone tissue by metastatic prostate cancer cells, comprising (a) obtaining osteogenic cell-conditioned medium; and (b) separating protein and non-protein components of the medium.

A further embodiment comprises methods of obtaining a protein factor produced by an osteogenic cell, wherein the factor promotes migration to and/or invasion of bone tissue by prostate cancer cells, comprising (a) obtaining osteogenic cell-conditioned medium; and (b) separating protein and non-protein components of the medium. Still a further embodiment comprises a method of separating a factor from osteogenic cell-conditioned medium, wherein the factor promotes migration to and/or invasion of bone tissue by cancer cells, comprising (a) obtaining osteogenic cell-conditioned medium; (b) fractionating components of osteogenic cell-conditioned medium; and (c) assaying for promotion of cancer cell migration and/or invasion in fractions from (b), wherein a fraction that promotes cancer cell migration and/or invasion contains separated factor.

In still a further embodiment, there are provided methods of identifying a factor from an osteogenic cell-conditioned medium, wherein the factor promotes migration to and/or invasion of bone tissue by prostate cancer cells, comprising (a) obtaining osteogenic cell-conditioned medium; (b) fractionating components of osteogenic cell-conditioned medium; (c) assaying for promotion of cancer migration and/or invasion in fractions from (b); and (d) identifying the factor in the fraction of (c).

In additional embodiments, polyclonal antisera against osteogenic cell-conditioned medium are provided. Also provided is a method of preparing an antibody population comprising (a) generating polyclonal antisera against osteogenic cell-conditioned medium; and (b) depleting the antisera of antibodies reactive with proteins found in the medium in the absence of osteogenic cells. Additionally, a method of preparing a hybridoma cell is provided, the method comprising (a) generating a hybridoma cell population secreting antibodies against osteogenic cell-conditioned medium; and (b) depleting the antisera of antibodies reactive with proteins found in the medium in the absence of osteogenic cells.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B—Osteoblast-induced Changes in the Invasiveness and Migration of Metastatic C42B CaP Cells.

FIG. 2—Few Osteoblast-Secreted Proteins Modulate CaP Cell Migration. bFGF, basic fibroblast growth factor; IL, interleukin; HGF, hepatocyte growth factor; TGF B1, transforming growth factor beta 1; PDGF, platelet derived growth factor; IGF, insulin-like growth factor; SDF, stromal derived growth factor.

### DESCRIPTION OF ILLUSTRATIVE EMBODIMENTS

Certain cancers, such as prostate, and breast, are characterized by metastases that induce localized bone formation. In the case of prostate cancer cells, certain of these cells produce a signal that induces cells to produce bone tissue, whereas the others often form bone-eroding (osteolytic) lesions. Thus, the identifying factors that result in the metastasis of cancer cells to bone would permit another point of possible intervention in osteogenic metastatic cancers.

The present invention concerns the observation that osteogenic cells, such as pre-osteoblasts and obsteoblasts, produce a factor or factors that stimulate the migration and/or invasion of bone tissue by cancer cells. The invention also provides for methods of purifying and identifying such factors, as well as conditioned medium, and poly- and monoclonal antibodies thereto. Also described are methods of identifying an inhibitor of such factors.

I. Culturing Osteogenic Cells to Prepare Conditioned Medium

Under normal culture conditions, osteogenic cells are grown in the presence of varying amounts of serum, and remain adherent to the culture dish, essentially growing as a two dimensional, planar sheet of cells. The in vitro expansion of these cells requires their release from the plastic by trypsin treatment and re-culturing. After 4 to 6 weeks, the cells are placed in media containing serum and higher levels of calcium and phosphate. Osteogenic cells grown in serum-free conditions undergo a distinctly different developmental pattern. This process requires the presence of TGF-β, or other osteogenic growth factors, added within the first 0 to 48 hours of culture.

Conditioned medium from human primary pre-osteoblast cells was prepared by plating 5×10⁵ cells in 10 ml culture medium (10% FBS DMEM medium) to 100 mm tissue culture dishes. Following 24 hrs to allow for adherence, the medium was removed and the cells washed three times with 10 ml/dish PBS. Washed cells were then incubated 72 hrs in 10 ml/dish serum-free DMEM medium supplemented with 1% (v/v) ITS+(Becton Dickenson, Bedford. Mass.) Medium collected after 72 hrs was filtered through 0.2 μm filter and used directly in in vitro invasion and migration assays with human prostate cancer cells.

II. Metastasis

A. Metastatic Prostate Cancer Cells

The majority (70%) of human prostatic adenocarcinomas arise in the peripheral zone of the prostate. As the tumor develops, it begins to spread locally into the periprostatic fat, seminal vesicles and regional lymph nodes, specifically the hypogastric and obturator nodes. Once the tumor has breached the vascular bed, cancer cells will spread through the circulation (hematogenously) to distant sites where they become lodged in the capillary bed of permissive organs and form secondary tumors or metastases. The most common site of prostate cancer (CaP) metastasis is the axial skeleton, specifically the pelvis, femur and vertebral column, with lesser bony involvement in the ribs and skull. Distant visceral metastases are less common but include the liver, lung, and the dura of the brain. In general, the metastatic spread of cancer cells is a highly selective, non-random process. Only those tumor cells that can both respond to and manipulate the surrounding organ environment will ultimately form a metastatic lesion within the organ. With regards to CaP cells, bone marrow containing trabecular bone represents a permissive environment that can support metastatic CaP cell growth and survival (Pazdur, 2002).

In accordance with the present invention, a variety of cancer cells may be utilized for the various claimed embodiments. In the first instance, metastatic prostate cancer cells can be used as targets for an OCM activity or activities. These cells are sensitive to OCM and changes in their migration and/or invasiveness indicate modulation of their metastatic ability. Likewise, non-metastatic prostate cancer cells are used to indicate activities in OCM that alter the transformation of prostate cancer cells into metastatic cells. Finally, normal non-cancerous prostate cells are used to monitor the actions of these activities on normal prostate tissue.

B. Measuring Cell Migration

Chemotaxtic migration assays were performed as described with modifications (Jeffers et al., 1996). Briefly, cells were harvested with trypsin, washed once with serum-free medium, and resuspended in serum-free DMEM medium to a concentration of 5×10⁴ viable cells/ml. 25,000 cells/0.5 ml were then added to 24-well cell culture inserts with a 3 mm pore size (Becton Dickinson, Bedford, Mass.). The inserts were placed in wells containing 0.5 ml of serum-free DMEM, 72 hr serum-free conditioned medium from human primary osteoblasts and incubated for 48 hrs in 5% CO₂-95% air at 37° C. At the end of the incubation period, non-invading cells were removed with a cotton swab from the top of the filter and the invading cells stained with Hema 3® solution (CMS, Houston, Tex.). The total number of invading cells/filter was quantified under 10× magnification. Heat inactivated osteoblast conditioned medium, transforming growth factor beta 1, and platelet-derived growth factor beta were included as negative controls of migration. To measure in vitro invasion, cells were plated instead to Matrigel-coated cell culture inserts (Becton Dickinson).

C. Measuring Invasion

In the context of the present invention, assays will be performed to assess tumor cell invasion of bone tissue. Such assays rely on model systems that measure cancer cell migration into artificial supports comprised of agents such as collagen, fibronectin, thrombospondin, vitronectin, laminin, or combinations thereof. One combination is represented by Matrigel and a composite of various extracellular proteins.

III. Osteogenic Precursor Cells

The present invention provides for the use of conditioned medium obtained from either bone precursor or mature bone cells (osteogenic) cells. As used herein, a bone precursor cell is any cell that is capable of differentiating or expanding into an osteoblast cell. The following section describes the characteristics of these cells. Osteogenic or precursor cells are derived from primary sources such as bone marrow or bone. In addition, cells can be derived from several different species, including cells of human, bovine, equine, canine, feline and murine origin.

A. Bone Precursor Cells (Osteoprogenitor Cells)

Human bone precursor cells are characterized as small-sized cells that express low amounts of bone proteins (osteocalcin, osteonectin, and alkaline phosphatase) and have a low degree of internal complexity (Long et al., 1995). When stimulated to differentiate, these preosteoblast cells become osteoblast in their appearance, size, antigenic expression, and internal structure. Although these cells are normally present at very low frequencies in bone marrow, a process for isolating these cells has been described (Long et al., 1995). U.S. Pat. No. 5,972,703 further describes methods of isolating and using bone precursor cells, and is specifically incorporated herein by reference.

B. Preosteoblasts

Preosteoblasts are intermediate between osteoprogenitor cells and osteoblasts. The show increasing expression of bone phenotypic markers such as alkaline phosphatase (Kale et al., 2000). They have a more limited proliferative capacity, but nonetheless continue to divide and produce more preosteoblasts or osteoblasts.

C. Osteoblasts

Osteoblasts are the most mature cells of the bone cell lineage. They are large cells, possessing a eccentric nucleus, and produce of the extracellular proteins required for bone formation. They can be obtained from bone as populations of both preosteoblasts and osteoblasts as described in U.S. Ser. No. 09/753,043, which is specifically incorporated herein by reference.

D. Osteoblast Accessory Cells

Bone precursor cells appear to require accessory cells for outgrowth in vitro. A distinct cell population of relatively low cellular complexity (or maturity) of intermediate size, and of high TGFβRII expression, was previously identified as such containing bone accessory cells. Cell sorting based on these characteristics produces a cell population that is enriched in bone accessory cells and shows an increase in specific activity in HBPC expansion assays. See U.S. Pat. No. 6,576,465.

Flow cytometry of purified accessory cells shows that these cells express little, if any, STRO-1 antigen, and also lack expression of P-selectin, E-selectin or L-selectin. Additionally, it seems that these cells do not express, or express very little of the cell/matrix protein osteocalcin or osteonectin. The cells further lack expression of CD3 (a marker for T-cells), CD56 (an NK-cell marker), CD68 (a macrophage marker), CD34 (a hematopoietic cell marker), and von Willibrand's factor (an endothelial cell marker). Thus, these cells are not T-cells, hematopoietic cells, macrophages, NK-cells or endothelial cells. The cells are, as stated above, characterized by high TGFβRII expression.

IV. Purification Methods

In accordance with the present invention, purified factors that induce migration to and/or invasion of bone tissue, and methods for separating, purifying and identifying factors are provided. Using the separative techniques described below, conditioned medium may be fractionated and the subsequent fractions tested for activity, such as metastasis-inducing activity.

In various embodiments of the present invention, one will desired to fractionate molecules from osteogenic cell-conditioned medium. Any technique may prove useful, and can include chemical methods, such as phase partitioning or precipitating (salting out), physical methods, such as chromatography, isoelectric focusing, centrifugation or electrophoresis, enzymatic methods (glycosylases, proteases, lipases, etc.), mass spectrometry, and even thermal (heating, freeze-thawing).

Any of a wide variety of chromatographic procedures may be employed according to the present invention. For example, thin layer chromatography, gas chromatography, high performance liquid chromatography, paper chromatography, affinity chromatography or supercritical flow chromatography may be used to effect separation of various chemical species.

Partition chromatography is based on the theory that if two phases are in contact with one another, and if one or both phases constitute a solute, the solute will distribute itself between the two phases. Usually, partition chromatography employs a column, which is filled with a sorbent and a solvent. The solution containing the solute is layered on top of the column. The solvent is then passed through the column, continuously, which permits movement of the solute through the column material. The solute can then be collected based on its movement rate. The two most common types of partition chromatograph are paper chromatograph and thin-layer chromatograph (TLC); together these are called adsorption chromatography. In both cases, the matrix contains a bound liquid. Other examples of partition chromatography are gas-liquid and gel chromatography.

Paper chromatography is a variant of partition chromatography that is performed on cellulose columns in the form of a paper sheet. Cellulose contains a large amount of bound water even when extensively dried. Partitioning occurs between the bound water and the developing solvent. Frequently, the solvent used is water. Usually, very small volumes of the solution mixture to be separated is placed at top of the paper and allowed to dry. Capillarity draws the solvent through the paper, dissolves the sample, and moves the components in the direction of flow. Paper chromatograms may be developed for either ascending or descending solvent flow. Two dimensional separations are permitted by changing the axis of migation 90° after the first run.

Thin layer chromatography (TLC) is very commonly used to separate lipids and, therefore, is considered a preferred embodiment of the present invention. TLC has the advantages of paper chromatography, but allows the use of any substance that can be finely divided and formed into a uniform layer. In TLC, the stationary phase is a layer of sorbent spread uniformly over the surface of a glass or plastic plate. The plates are usually made by forming a slurry of sorbent that is poured onto the surface of the gel after creating a well by placing tape at a selected height along the perimeter of the plate. After the sorbent dries, the tape is removed and the plate is treated just as paper in paper chromatography. The sample is applied and the plate is contacted with a solvent. Once the solvent has almost reached the end of the plate, the plate is removed and dried. Spots can then be identified by fluorescence, immunologic identification, counting of radioactivity, or by spraying varying reagents onto the surface to produce a color change.

In Gas-Liquid chromatography (GLC), the mobile phase is a gas and the stationary phase is a liquid adsorbed either to the inner surface of a tube or column or to a solid support. The liquid usually is applied as a solid dissolved in a volatile solvent such as ether. The sample, which may be any sample that can be volatized, is introduced as a liquid with an inert gas, such as helium, argon or nitrogen, and then heated. This gaseous mixture passes through the tubing. The vaporized compounds continually redistribute themselves between the gaseous mobile phase and the liquid stationary phase, according to their partition coefficients.

The advantage of GLC is in the separation of small molecules. Sensitivity and speed are quite good, with speeds that approach 1000 times that of standard liquid chromatography. By using a non-destructive detector, GLC can be used preparatively to purify grams quantities of material. The principal use of GLC has been in the separation of alcohols, esters, fatty acids and amines.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

The gel material for gel chromatography is a three-dimensional network whose structure is usually random. The gels consist of cross-linked polymers that are generally inert, do not bind or react with the material being analyzed, and are uncharged. The space filled within the gel is filled with liquid and this liquid occupies most of the gel volume. Common gels are dextran, agarose and polyacrylamide; they are used for aqueous solution.

High Performance Liquid Chromatography (HPLC) is characterized by a very rapid separation with extraordinary resolution of peaks. This is achieved by the use of very fine particles and high pressure to maintain and adequate flow rate. Separation can be accomplished in a matter of minutes, or a most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

V. Antibodies

In one embodiment of the present invention, one will desire to prepare antibodies against the factor or factors contained in osteogenic precursor cell-conditioned medium. An antibody can be a polyclonal or a monoclonal antibody (Mab). In one embodiment, an antibody is a monoclonal antibody. Means for preparing and characterizing antibodies are well known in the art (see, e.g., Harlow and Lane, 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH), Multiple Antigenic Peptide (MAP), or bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition. The immunizing composition is administered in a manner effective to stimulate antibody producing cells. Rodents such as mice and rats are preferred animals, however, the use of rabbit, sheep, and frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag4 1, Sp210-Ag14, FO, NSO/U, MPC-11, MPC11-X45-GTG 1.7 and S194/5XX0 Bul; for rats, one may use R210.RCY3, Y3-Ag1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al. (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

VI. Screening Assays

In still further embodiments, the present invention provides method for identifying inhibitors of metastatic factors. These assays may comprise random screening of large libraries of candidate substances; alternatively, the assays may be used to focus on particular classes of compounds selected with an eye towards attributes that are believed to make them more likely to modulate the function of the metastatic factors identified herein.

To identify an inhibitor, one generally will determine the migration and/or invasion of a cancer cell in the presence and absence of the candidate substance. For example, a method generally comprises:
(a) providing a candidate modulator;
(b) admixing the candidate modulator with a cell;
(c) measuring migration and/or invasion of the cell in step (c); and
(d) comparing the migration and/or invasion in step (c) with the migration and/or invasion in the absence of said candidate modulator,
wherein a reduction between the migration and/or invasion indicates that said candidate modulator is, indeed, an inhibitor of metastasis.
   Assays may be conducted with isolated cells or intact organisms.

It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate substance” refers to any molecule that may potentially inhibit metastatic (migration and/or invasion) activity. The candidate substance may be a protein or fragment thereof, a small molecule, or even a nucleic acid molecule. It also is possible to use antibodies as inhibitors, which may then yield a pharmacore upon which future drug design can be based.

One may also acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

B. In Cyto Assays

The present invention contemplates the screening of compounds for their ability to inhibit metastatic (migration and/or invasion) activity. Various cells can be utilized for such screening assays, including those cancers cells discussed elsewhere herein. The cell is examined for metastatic (migration and/or invasion) activity. Alternatively, molecular analysis may be performed, for example, looking at protein expression, mRNA expression (including differential display of whole cell or polyA RNA) and others.

C. In Vivo Assays

In vivo assays involve the use of various animal models. Due to their size, ease of handling, and information on their physiology and genetic make-up, mice are a preferred embodiment. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for inhibitors may be conducted using an animal model derived from any of these species.

In such assays, one or more candidate substances are administered to an animal, and the ability of the candidate substance(s) to alter metastatic (migration and/or invasion) activity of cancer cells, as compared to a similar animal not treated with the candidate substance(s), identifies an inhibitor modulator.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in cyto assays.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

The molecular mechanisms mediating the bone specific metastasis of prostate cancer cells (CaP) are unclear, but may involve the selective homing CaP cells to bone along an osteoblast-derived chemotaxtic gradient. The inventors used human primary preosteoblasts and osteoblasts (OB) to directly test the role of OB-secreted proteins on CaP cell in vitro migration and invasion.

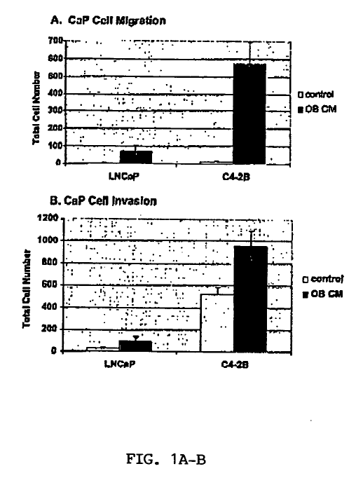

The data show that conditioned medium (CM) from human primary OB secrete a potent chemotaxtic factor that stimulated the selective migration and invasion of bone metastatic LNCaP-C4-2B and VCaP human CaP cells versus parental LNCaP or brain metastatic DuCaP cells (latter not shown; FIGS. 1A-B). Thus, a 7- to 8-fold increase in cell migration is seen following exposure to OB-CM (FIG. 1A), as is an approximate two-fold increase in invasive potential (FIG. 1B).

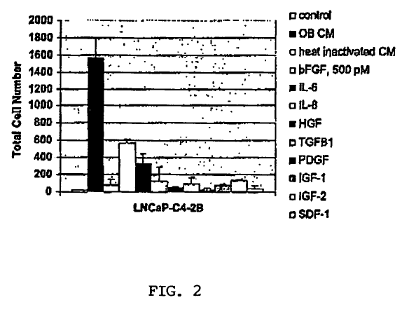

The chemotaxtic effect could be completely abrogated by boiling the CM (FIG. 2) thus indicating that the activity in OB-CM is a protein. Likewise, proteases abolish the activity (not shown). DNA microarray analysis of proteins secreted by osteoblasts show the presence of 8 proteins capable of influencing CaP cell function: Hepatocyte Growth Factor, Insulin like Growth Factors I and II, Basic Fibroblast Growth Factor, Stromal Derived Growth Factor 1, Vascular Endothelial Growth Factor, Interleukin 6, and Interleukin 8. However, evaluation of these proteins in migration assays demonstrated that all but two of these failed to increase CaP cell migration (FIG. 2). Importantly, neutralizing antibodies to each of the two stimulatory proteins, basic Fibroblast Growth Factor and IL-6, failed to block OB-CM induced migration (not shown).

Thus, osteoblasts secrete a seemingly unique factor that affects the metastatic potential of CaP cells. Taken together, the data show that osteoblast-derived proteins stimulate the migration and invasiveness of bone metastatic CaP cells, which may explain the high prevalence with which CaP metastasizes to the bone.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 1,995,970
U.S. Pat. No. 2,676,945
U.S. Pat. No. 2,703,316
U.S. Pat. No. 2,758,987
U.S. Pat. No. 2,951,828
U.S. Pat. No. 3,531,561
U.S. Pat. No. 4,196,265
U.S. Pat. No. 4,352,883
U.S. Pat. No. 4,443,546
U.S. Pat. No. 4,533,637
U.S. Pat. No. 5,063,157
U.S. Pat. No. 5,405,772
U.S. Pat. No. 5,972,703
U.S. Ser. No. 09/753,043
Cynamid Research Develops World's First Synthetic Absorbable Suture, Chemistry and Industry, 905, 1970.
Allcock and Fuller, J. Am. Chem. Soc., 103:2250-2256, 1981.
Barnes and Sam, “Serum-free cell culture: A unifying approach,” Cell, 22:649-655, 1980.
Barnes, “Serum-free animal cell culture,” Bio. Techniques, 5: 534-542, 1987.
Bonadio and Goldstein, In: Molecular and Cellular Biology of Bone, Noda (Ed.), Academic Press, Inc., San Diego, 169-189, 1993.
Broad et al., Cytotechnology 5:47-55, 1991.
Byers and Steiner, Annu. Rev. Med., 43:269-289, 1992.
Campbell, In: Monoclonal Antibody Technology, Laboratory Techniques in Biochemistry and Molecular Biology, Burden and Von Knippenberg (Eds.), Elseview, Amsterdam, 13:71-74/75-83, 1984.
Costantino et al., Arch. Otolaryngol. Head Neck Surg., 117(4):379-384, 1991.
Elgendy et al., Biomaterials, 14:263-269, 1993.
EP 481 791
Festuccia et al., Oncology Res., 9(8):419-31, 1997.
Frame, Int. J Oral Maxillofac. Surg., 16(6):642-655, 1987.
Friedman et al., Arch. Otolaryngol. Head Neck Surg., 117(4), 386-389, 1991.
Gefter et al., Somatic Cell Genet., 3:231-236, 1977.
Glowacki et al., Clin. Plast. Surg., 12(2), 233-241, 1985.
Goding, In: Monoclonal Antibodies: Principles and Practice, 2d ed., Academic Press, FL., 60-66, and 71-74, 1986.
Harada, Shikwa-Gakuho, 89(2):263-297, 1989.
Harlow and Lane, Antibodies “A Laboratory Manual, Cold Spring Harbor Laboratory, 1988.
Hollinger and Battistone, Clinical Orthop. Rel. Res., 207:290-305, 1986.
Jayme, Cytotechnology 5(1):15-30, 1991.
Jeffers et al., Mol. Cell Biol. 16(3):1115-1125, 1996.
Kale et al., Nat. Biotech., 18:954-958, 2000.
Kimura et al., Urologia Internationalis., 48(1):25-30, 1992.
Kohler and Milstein, Eur. J Immunol., 6:511-519, 1976.
Kohler and Milstein, Nature, 256:495-497, 1975.
Kulkarni et al., J. Biom. Mater. Res., 5:169-81, 1971.
Laurencin et al., J. Biom. Mater. Res., 27(7):963-973, 1993.
LeRoy et al., Prostate, 50(2):104-111, 2002
Long, J. Clin. Invest., 95:881-887, 1995.
Marden et al., J. Craniofac. Surg., 1(3):154-160, 1990.
Martinez et al., J., Cellular Biochem., 61(1):18-25, 1996.
Ohgushi et al., Acta Orthop. Scand., 60(3):334-339, 1989.
Ono et al., Biomaterials, 11(4):265-271, 1990.
Parsons et al., Ann. NY Acad. Sci., 523:190-207, 1988.
Passuti et al., Clin. Orthop., 248:169-176, 1989.
Pazdur, In: Cancer Management: A Multidisciplinary Approach, PPR Inc., NY, 2002.
Pinholt et al., J. Oral Maxillofac. Surg., 50(8):859-867, 1992.
Pinholt et al., Scand. J Dent. Res., 99(2):154-161, 1991.
Pochon et al., Z-Kinderchir., 41(3):171-173, 1986.
Prockop, J. Biol. Chem., 265:15349-15352, 1990.
Roesgen, Unfallchirurgle, 16(5):258-265, 1990.
Santibanez et al. Brit. J. Cancer, 74(3):418-22, 1996
Toriumi et al., Arch. Otolaryngol Head Neck Surg., 117:1101-1112, 1991.
Wade et al., In: Organomet. Polym., Carraher et al. (Eds.), Academic Press, NY, 283-288, 1978.
WO 95/06112

What is claimed is:

1. A method of identifying an agent useful in preventing cancer cell metastasis to bone comprising:
(a) providing osteogenic precursor cell-conditioned medium (OCM);
(b) providing a bone-metastatic cancer cell;
(c) contacting said OCM with said bone-metastatic cancer cell in the presence of a candidate substance; and
(d) assessing the migration and/or invasion of said bone-metastatic cancer cell,
wherein a difference in the migration and/or invasion seen in step (d), as compared to the migration and/or invasion seen in the absence of said candidate substance, identifies said candidate substance as a bone metastasis inhibitor.

2. The method of claim 1, wherein said candidate substance is an organopharmaceutical small molecule.

3. The method of claim 1, wherein said candidate substance is an antibody or fragment thereof.

4. The method of claim 1, wherein said candidate substance is peptide, a polypeptide, or peptidomimetic.

5. The method of claim 1, wherein said bone-metastatic cancer cell is a prostate cancer cell or a breast cancer cell.

6. The method of claim 5, wherein said prostate cancer cell is a LNCaP-C4-2B cell.

7. The method of claim 1, wherein said OCM is conditioned with a preosteoblast cell, an osteoblast cell, and osteoblast precursor cell and/or an osteoblast accessory cell.

8. The method of claim 1, wherein invasion is monitored by cell passage through specific extracellular proteins such as collagen, fibronectin, thrombospondin, vitronectin, laminin or combinations thereof.

9. The method of claim 1, wherein migration is monitored by cell passage through membranes with pore sizes of about 3 to about 8 microns, or about 3 microns.

10. The method of claim 1, further comprising performing step (d) in the absence of said candidate substance.

11. A composition comprising medium conditioned by growth of an osteogenic cell therein.

12. A heat-labile protein factor obtained from osteogenic cell-conditioned medium that promotes migration to and/or invasion of bone tissue by prostate cancer cells.

13. A protein factor obtained from osteogenic cell-conditioned medium that promotes migration to and/or invasion of bone tissue by metastatic prostate cancer cells.

14. A method of obtaining a protein factor produced by an osteogenic cell, wherein said factor promotes migration to and/or invasion of bone tissue by metastatic prostate cancer cells, comprising:
(a) obtaining osteogenic cell-conditioned medium; and
(b) separating protein and non-protein components of said medium.

15. A method of obtaining a protein factor produced by an osteogenic cell, wherein said factor promotes migration to and/or invasion of bone tissue by prostate cancer cells, comprising:
(a) obtaining osteogenic cell-conditioned medium; and
(b) separating protein and non-protein components of said medium.

16. A method of separating a factor from osteogenic cell-conditioned medium, wherein said factor promotes migration to and/or invasion of bone tissue by cancer cells, comprising:
(a) obtaining osteogenic cell-conditioned medium;
(b) fractionating components of osteogenic cell-conditioned medium; and
(c) assaying for promotion of cancer cell migration and/or invasion in fractions from (b),
wherein a fraction that promotes cancer cell migration and/or invasion contains separated factor.

17. A method of identifying a factor from an osteogenic cell-conditioned medium, wherein said factor promotes migration to and/or invasion of bone tissue by prostate cancer cells, comprising:
(a) obtaining osteogenic cell-conditioned medium;
(b) fractionating components of osteogenic cell-conditioned medium;
(c) assaying for promotion of cancer migration and/or invasion in fractions from (b); and
(d) identifying the factor in the fraction of (c).

18. Polyclonal antisera against osteogenic cell-conditioned medium.

19. A method of preparing an antibody population comprising:
(a) generating polyclonal antisera against osteogenic cell-conditioned medium; and
(b) depleting said antisera of antibodies reactive with proteins found in the medium in the absence of osteogenic cells.

20. A method of preparing a hybridoma cell comprising:
(a) generating a hybridoma cell population secreting antibodies against osteogenic cell-conditioned medium; and
(b) depleting said antisera of antibodies reactive with proteins found in the medium in the absence of osteogenic cells.
